(19) United States
(12) Patent Application Publication
(10) Pub. No.: US 2020/0119359 A1
(43) Pub. Date: Apr. 16, 2020

(54) OLIGOMER AND LITHIUM BATTERY

(71) Applicant: National Taiwan University of Science and Technology, Taipei (TW)

(72) Inventors: Chorng-Shyan Chern, Taipei (TW); Fu-Ming Wang, Taipei (TW); Quoc Thai Pham, Taipei (TW)

(73) Assignee: National Taiwan University of Science and Technology, Taipei (TW)

(21) Appl. No.: 16/158,304

(22) Filed: Oct. 12, 2018

(51) Int. Cl. H01M 4/62; H01M 10/0525; C08G 61/12; C08G 73/10; C08G 83/00; C08F 22/10; C07D 207/416; C07D 403/14; C07D 233/78

(52) U.S. Cl. CPC H01M 4/628; H01M 10/0525; C08G 61/124; C08G 73/1092; H01M 2004/028; C08F 22/10; C07D 207/416; C07D 403/14; C07D 233/78; C08G 83/005

(57) ABSTRACT

A oligomer and a lithium battery are provided. The oligomer is obtained by a polymerization reaction of a compound containing at least one ethylenically unsaturated group and a nucleophile compound. The compound containing at least one ethylenically unsaturated group is selected from a group consisting of a maleimide-based compound, an acrylate ester-based compound, a methacrylate ester-based compound, an acrylamide-based compound, a vinylamide-based compound and a combination thereof. The nucleophile compound is selected from a group consisting of monomaleimide, trithiocyanuric acid, hydantoin, hydantoin derivative, thiohydantoin, thiohydantoin derivative and a combination thereof.

### BACKGROUND

### Technical Field

The invention relates to an oligomer and a battery, and more particularly, to an oligomer for a lithium battery and a lithium battery.

### Description of Related Art

Since primary batteries are not enviroment-friendly, the market demand for secondary lithium batteries with characteristics such as rechargeability, light weight, high voltage value, and high energy density has been growing in recent years. As a result, current performance requirements for the secondary lithium battery such as lightweight, durability, high voltage, high energy density, and high safety have also become higher. In particular, secondary lithium batteries have relatively high potential in the application and expandability in light electric vehicles, electric vehicles, and the large power storage industry.

However, among the commercialized secondary lithium batteries in the general market, since lithium transition metal oxide is used as the cathode, the cathode readily reacts with the electrolyte solution in high temperature applications and becomes damaged. As a result, oxygen in the lithium metal oxide is released and becomes part of a combustion reaction. This is one of the main causes for the explosion, swelling, and performance degradation of the secondary lithium battery. Therefore, continuously maintaining the structural stability and high performance of the cathode material in high temperature applications is one of the desired goals of those skilled in the art.

### SUMMARY

The invention provides an oligomer that can be applied in the cathode material of a lithium battery such that the lithium battery has good performance.

The invention provides a lithium battery having the oligomer.

The oligomer of the invention is obtained a polymerization reaction of a compound containing at least one ethylenically unsaturated group and a nucleophile compound, wherein the compound containing at least one ethylenically unsaturated group is selected from a group consisting of a maleimide-based compound, an acrylate ester-based compound, a methacrylate ester-based compound, an acrylamide-based compound, a vinylamide-based compound and a combination thereof, and the nucleophile compound is selected from a group consisting of monomaleimide (MI), trithiocyanuric acid (TCA), hydantoin (HD), hydantoin derivative, thiohydantoin (THD), thiohydantoin derivative and a combination thereof.

In an embodiment of the oligomer of the invention, wherein a mole ratio of the compound containing at least one ethylenically unsaturated group and the nucleophile compound is, for example, between 1:5 and 5:1.

In an embodiment of the oligomer of the invention, wherein the maleimide-based compound includes, for example, monomaleimide or bismaleimide (BMI).

In an embodiment of the oligomer of the invention, wherein the acrylate ester-based compound includes, for example, bisphenol A diacrylate (BADA) or bisphenol A ethoxylate diacrylate (BEDA).

In an embodiment of the oligomer of the invention, wherein the methacrylate ester-based compound includes, for example, bisphenol A dimethacrylate (BMA).

In an embodiment of the oligomer of the invention, wherein the acrylamide-based compound includes, for example, bisacrylamide (BA).

In an embodiment of the oligomer of the invention, wherein the vinylamide-based compound includes, for example, N-vinylformamide (NVF) or N-vinylacetamide (NVA).

In an embodiment of the oligomer of the invention, wherein a reaction temperature of the polymerization reaction is, for example, between 25° C. and 200° C.

A lithium battery of the invention includes an anode, a cathode, a separator, an electrolyte solution, and a package structure. The cathode and the anode are separately disposed, and the cathode includes the oligomer. The separator is disposed between the anode and the cathode, and the separator, the anode, and the cathode define a housing region. The electrolyte solution is disposed in the housing region. The package structure covers the anode, the cathode, and the electrolyte solution.

In an embodiment of the lithium battery of the invention, the electrolyte solution includes an organic solvent, a lithium salt, and an additive, wherein the additive is, for example, monomaleimide, polyrnaleimide, bismaleimide, polybismaleimide, a copolymer of bismaleimide and monomaleimide, vinylene carbonate, or a mixture thereof.

Based on the above, by using the compound containing at least one ethylenically unsaturated group and the nucleophile compound to prepare the oligomer of the invention, the oligomer of the invention can be applied in the cathode material of a lithium battery, such that the lithium battery still has good capacitance, battery efficiency, and charge and discharge cycle life even in high-temperature operation.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a cross-sectional schematic of a lithium battery according to an embodiment of the invention.

FIG. 2 shows a diagram illustrating the relationship between the number of charge and discharge cycles and discharge capacity of the lithium battery of examples of the invention and comparative examples at room temperature.

### DESCRIPTION OF THE EMBODIMENTS

In the present specification, a range represented by “a numerical value to another numerical value” is a schematic representation for avoiding listing all of the numerical values in the range in the specification. Therefore, the recitation of a specific numerical range covers any numerical value in the numerical range and a smaller numerical range defined by any numerical value in the numerical range, as is the case with any numerical value and the smaller numerical range in the specification.

Moreover, in the present specification, skeleton formulas are sometimes used to represent compound structures. Such representation can omit carbon atoms, hydrogen atoms, and carbon-hydrogen bonds. Of course, structural formulas with clear illustrations of functional groups are definitive.

To prepare an oligomer that can be applied in the cathode material of a lithium battery such that the lithium battery still has good performance in a high-temperature environment, the invention provides an oligomer that can achieve the advantages above. In the following, embodiments are provided as examples of actual implementation of the invention.

An embodiment of the invention provides an oligomer. The oligomer is obtained by the polymerization reaction of a compound containing at least one ethylenically unsaturated group and a nucleophile compound.

In the present specification, the compound containing at least one ethylenically unsaturated group is selected from a group consisting of a maleimide-based compound, an acrylate ester-based compound, a methacrylate ester-based compound, an acrylamide-based compound, a vinylamide-based compound and a combination thereof. The maleimide-based compound can be monomaleimide or bismaleimide. The acrylate ester-based compound can be bisphenol A diacrylate or bisphenol A ethoxylate diacrylate. The methacrylate ester-based compound can be bisphenol A dimethacrylate. The acrylamide-based compound can be bisacrylamide. The vinylamide-based compound can be N-vinylformamide or N-vinylacetamide.

In the present specification the nucleophile compound is selected from a group consisting of monomaleimide, trithiocyanuric acid, hydantoin, hydantoin derivative, thiohydantoin, thiohydantoin derivative and a combination thereof. The monomaleimide may also be known as 2,5-pyrrolidone.

In an embodiment of the invention, the oligomer is obtained by reacting a compound containing at least one ethylenically unsaturated group and a nucleophile compound in a solvent. More specifically, a polymerization reaction can be performed by reacting the compound containing at least one ethylenically unsaturated group and the nucleophile compound in the solvent by a Michael addition reaction or a free radical copolymerization reaction. In an embodiment of the invention, the molar ratio of the compound containing at least one ethylenically unsaturated group and the nucleophile compound is, for example, between 1:5 and 5:1. If the molar ratio of the compound containing at least one ethylenically unsaturated group and the nucleophile compound is less than 1:5, then reactivity is poor. If the molar ratio of the compound containing at least one ethylenically unsaturated group and the nucleophile compound is higher than 5:1, then an electrochemical side reaction readily occurs. The temperature of the polymerization reaction is, for example, between 25° C. and 200° C., and the reaction time is, for example, between 0.5 hours and 8 hours.

The solvent can be an organic solvent, such as (but not limited to) N-methyl pyrollidone (NMP), dimethylformamide (DMF), dimethyl sulfoxide (DMSO) or dimethylacetamide (DMAC). The solvent can be used alone or in combination.

The Michael addition reaction can also be performed in the presence of a catalyst, i.e. the compound containing at least one ethylenically unsaturated group, the nucleophile compound, and the catalyst are dissolved in the solvent for the reaction. At this point, the reaction temperature is, for example, between, 25° C. and 80° C., the reaction time is, for example, between 0.5 hours and 2 hours, the catalyst is, for example, triethylamine or triphenylphosphine (TPP), and the content of the catalyst is, for example, 1 part by weight to 10 parts by weight.

In the following, the Michael addition reaction and the free radical (co)polymerization reaction for the compound containing at least one ethylenically unsaturated group and the nucleophile compound are described. The exemplary examples are not used to limit present invention.

### EXEMPLARY EXAMPLE 1

MI is used as the compound containing at least one ethylenically unsaturated group and the nucleophile compound at the same time. The Michael addition reaction and the free radical polymerization reaction are performed by using MI to form the oligomer of the present invention. That is, a self-polymerization reaction of MI is performed to form the oligomer of the present invention.

Michael addition reaction:

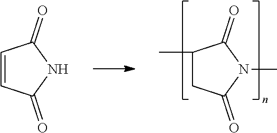

free radical polymerization reaction:

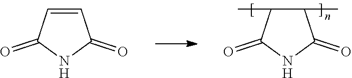

### EXEMPLARY EXAMPLE 2

BMI is used as the compound containing at least one ethylenically unsaturated group and MI is used as the nucleophile compound. The Michael addition reaction and the free radical copolymerization reaction are performed by using BMI and MI to form the oligomer of the present invention.

Michael addition reaction:

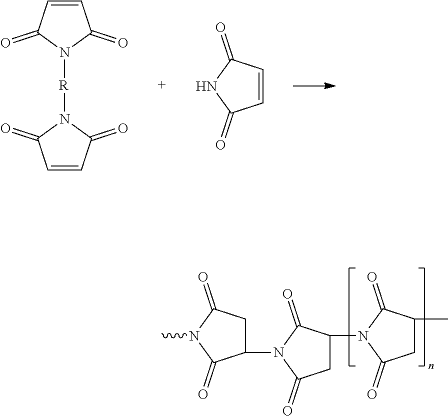

free radical copolymerization reaction:

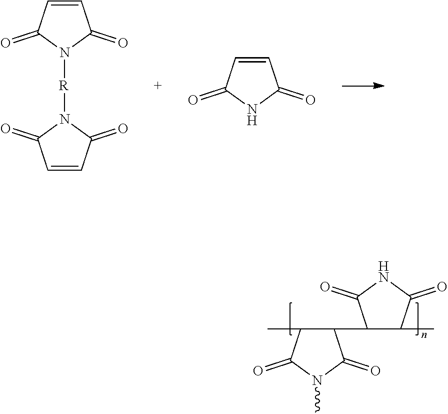

### EXEMPLARY EXAMPLE 3

BMI is used as the compound containing at least one ethylenically unsaturated group and TCA is used as the nucleophile compound. The Michael addition reaction is performed by using BMI and TCA to form the oligomer of the present invention.

Michael addition reaction:

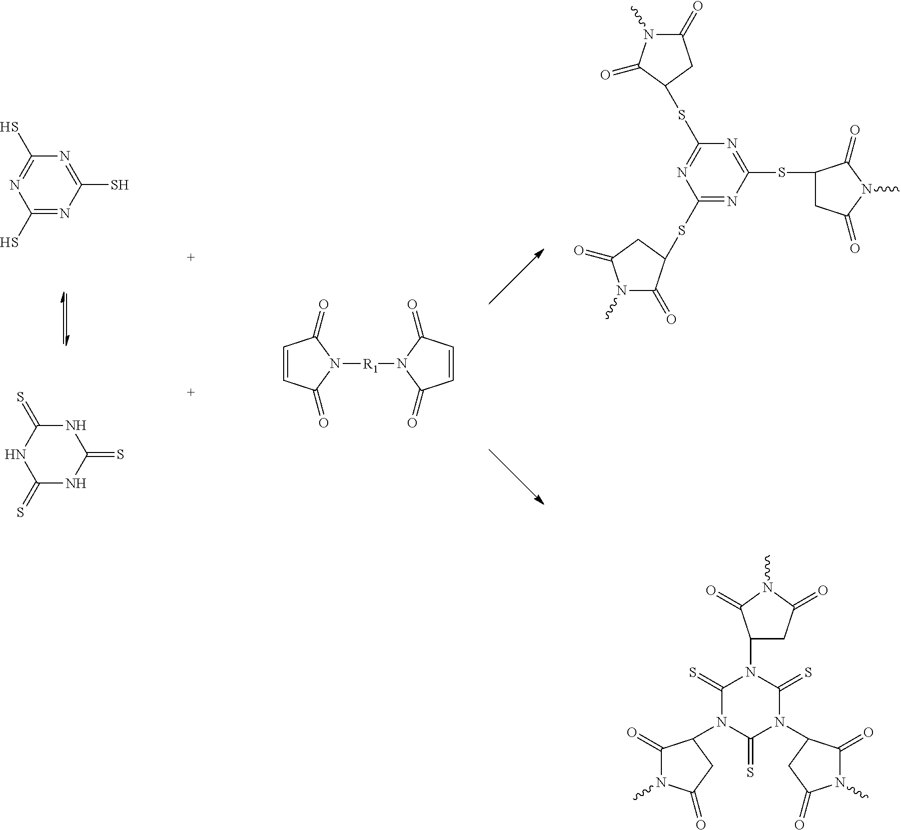

### EXEMPLARY EXAMPLE 4

BMI is used as the compound containing at least one ethylenically unsaturated group and HD is used as the nucleophile compound. The Michael addition reaction and the free radical polymerization reaction are performed by using BMI and HD to form the oligomer of the present invention.

Michael addition reaction:

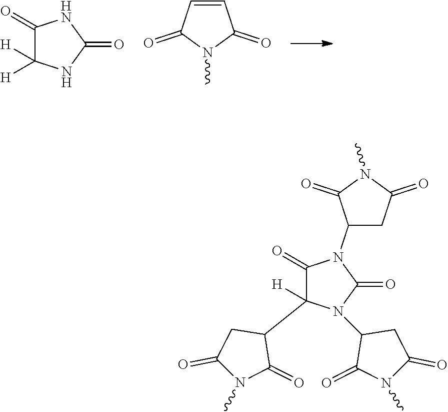

free radical polymerization reaction:

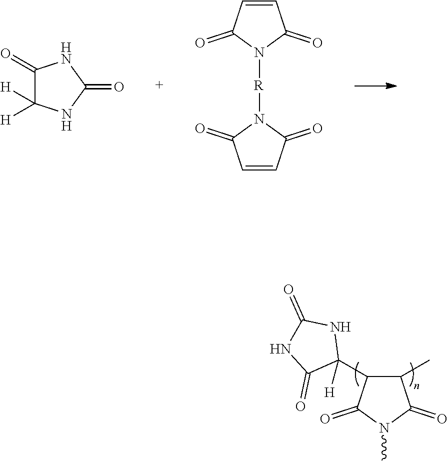

### EXEMPLARY EXAMPLE 5

BMI is used as the compound containing at least one ethylenically unsaturated group and THD is used as the nucleophile compound. The Michael addition reaction and the free radical polymerization reaction are performed by using BMI and THD to form the oligomer of the present invention.

Michael addition reaction:

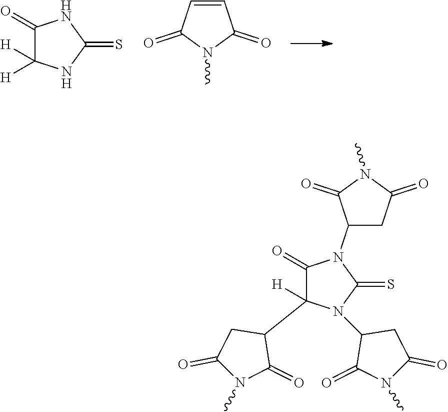

free radical polymerization reaction:

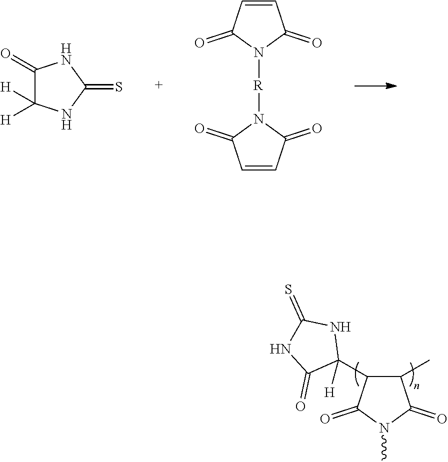

### EXEMPLARY EXAMPLE 6

BMA is used as the compound containing at least one ethylenically unsaturated group and MI is used as the nucleophile compound. The Michael addition reaction and the free radical copolymerization reaction are performed by using BMA and MI to form the oligomer of the present invention.

Michael addition reaction:

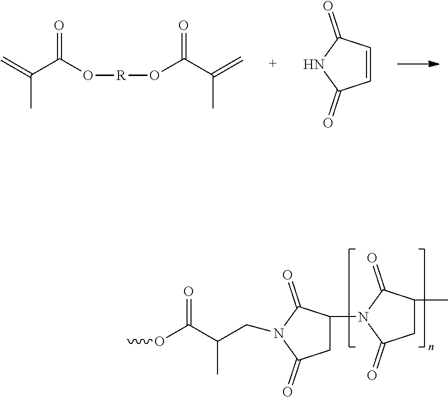

free radical copolymerization reaction:

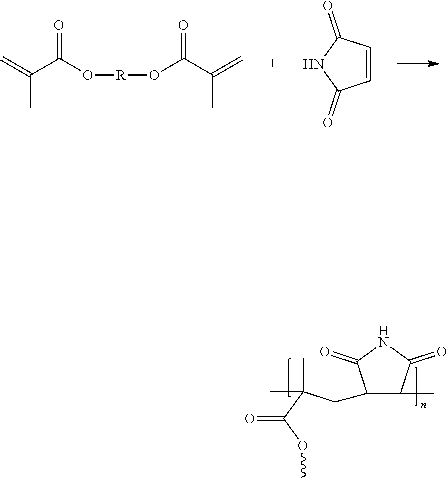

### EXEMPLARY EXAMPLE 7

BMA is used as the compound containing at least one ethylenically unsaturated group and HD is used as the nucleophile compound. The Michael addition reaction and the free radical polymerization reaction are perfoimed by using BMA and HD to form the oligomer of the present invention.

Michael addition reaction:

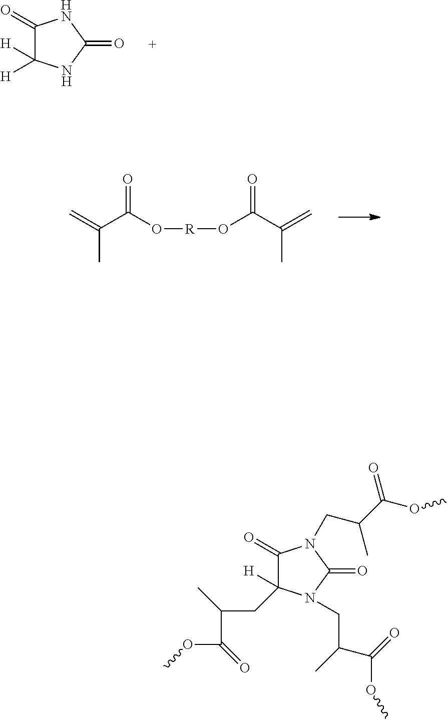

free radical polymerization reaction:

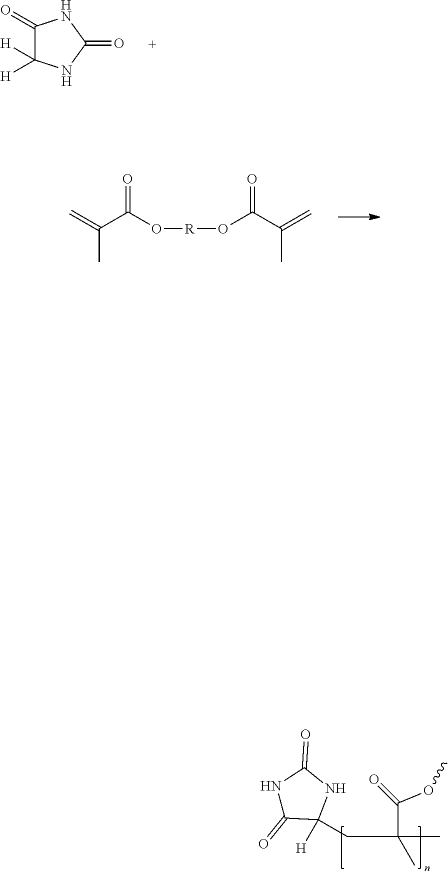

### EXEMPLARY EXAMPLE 8

BA is used as the compound containing at least one ethylenically unsaturated group and MI is used as the nucleophile compound. The Michael addition reaction and the free radical copolymerization reaction are performed by using BA and MI to form the oligomer of the present invention.

Michael addition reaction:

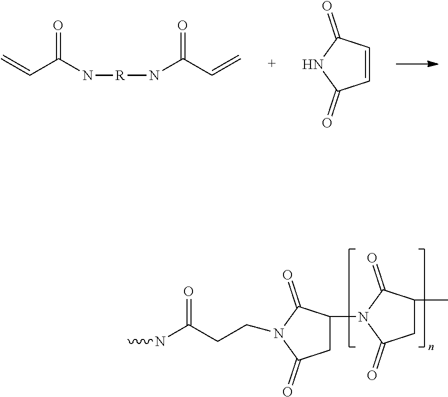

free radical copolymerization reaction:

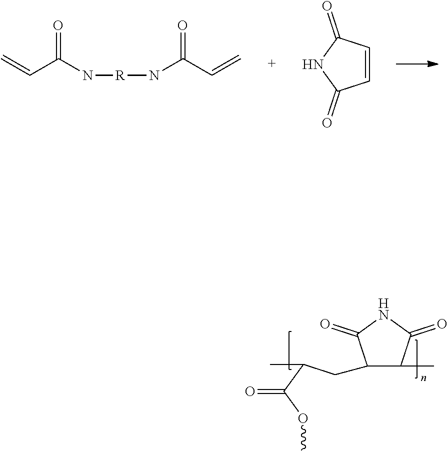

### EXEMPLARY EXAMPLE 9

BA is used as the compound containing at least one ethylenically unsaturated group and TCA is used as the nucleophile compound. The Michael addition reaction is perfonned by using BA and TCA to form the oligomer of the present invention.

Michael addition reaction:

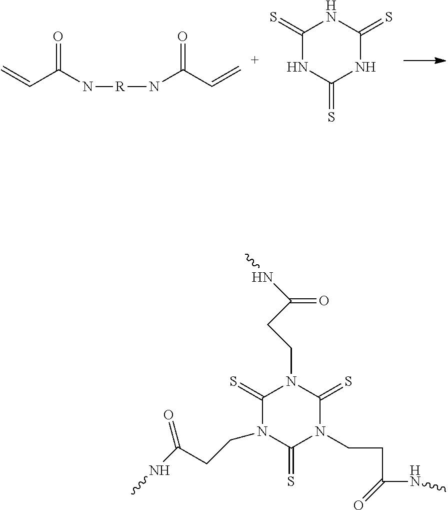

In the embodiments of the invention, the oligomer has a hyperbranched structure. “Hyperbranched structure” is a structure formed by adding the nucleophile compound on the carbon-carbon double bonds of the compound containing at least one at least one ethylenically unsaturated group group such that the carbon-carbon double bonds of the compound containing at least one at least one ethylenically unsaturated group group can be opened up, thereby allowing the two carbon atoms or one of the two carbon atoms to bond with other atoms of the nucleophile compound for branching and ordering polymerization reactions by using the compound containing at least one at least one ethylenically unsaturated group group as an architecture matrix during the addition polymerization reaction of the compound containing at least one at least one ethylenically unsaturated group group and the nucleophile compound.

The oligomer of the invention can be applied in the cathode material of a lithium battery. More specifically, the oligomer of the invention has good thermal reactivity, and therefore forms a protective layer on the surface of the cathode material to effectively block damage to the cathode structure in a high-temperature environment. The reasons are as follows: the resulting oligomer has a highly-branched structure and can therefore form a stable organic polymer with the metal oxide in a regular cathode material, and the oligomer has high thermal reactivity, high stability, and a rigid chemical structure, and therefore can provide high thermal stability to the resulting protective layer. As a result, the lithium battery having a cathode material including the oligomer of the invention can have good capacitance, battery efficiency, and safety in a high-temperature environment, and have excellent battery cycle life.

In the following, the lithium battery including the oligomer of the invention is described.

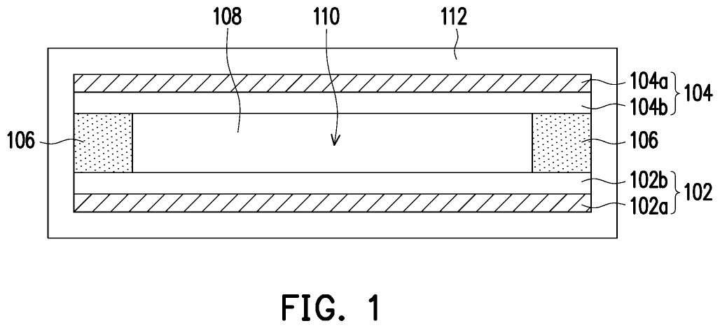

FIG. 1 is a cross-sectional schematic diagram of a lithium battery according to an embodiment of the invention. Referring to FIG. 1, a lithium battery 100 includes an anode 102, a cathode 104, a separator 106, an electrolyte solution 108, and a package structure 112.

The anode 102 includes an anode metal foil 102a and an anode material 102b, wherein the anode material 102b is disposed on the anode metal foil 102a through coating or sputtering. The anode metal foil 102a is, for example, a copper foil, an aluminum foil, a nickel foil, or a high-conductivity stainless steel foil. The anode material 102b is, for example, carbide or metal lithium. The carbide is, for example, carbon powder, graphite, carbon fiber, carbon nanotube, graphene, or a mixture thereof. However, in other embodiments, the anode 102 can also only include the anode material 102b.

The cathode 104 and the anode 102 are separately disposed. The cathode 104 includes a cathode metal foil 104a and a cathode material 104b, wherein the cathode material 104b is disposed on the cathode metal foil 104a through coating. The cathode metal foil 104a is, for example, a copper foil, an aluminum foil, a nickel foil, or a high-conductivity stainless steel foil.

The cathode material 104b includes the oligomer of the invention and a lithium-mixed transition metal oxide. The lithium-mixed transition metal oxide is, for example, LiMnO₂, LiMn₂O₄, LiCoO₂, Li₂Cr₂O₇, Li₂CrO₄, LiNiO₂, LiFeO₂, LiNiₓCo₁₋ₓO₂, LiFePO₄, LiMn_{0.5}Ni_{0.5}O₂, LiMn_{1/3}Co_{1/3}Ni_{1/3}O₂, LiMc_{0.5}Mn_{1.5}O₄, or a combination thereof, wherein 0<x<1 and Mc is a divalent metal.

Based on a total weight of 100 parts by weight of the cathode material 104b, the content of the oligomer is 0.5 parts by weight to 5 parts by weight (preferably 1 part by weight to 3 parts by weight). The content of the lithium-mixed transition metal oxide is, for example, 80 parts by weight to 95 parts by weight. If the content of the oligomer is less than 0.5 parts by weight, then the battery safety characteristic is not significant; and if the content of the oligomer is higher than 5 parts by weight, then battery cycle life is poor.

Moreover, the lithium battery 100 can further include a polymer binder. The polymer binder reacts with the anode 102 and/or the cathode 104 to increase the mechanical properties of the electrode(s). Specifically, the anode material 102b can be adhered to the anode metal foil 102a through the polymer binder, and the cathode material 104b can be adhered to the cathode metal foil 104a through the polymer binder. The polymer binder is, for example, polyvinylidene difluoride (PVDF), styrene-butadiene rubber (SBR), polyamide, melamine resin, or a combination thereof.

The separator 106 is disposed between the anode 102 and the cathode 104, and the separator 106, the anode 102, and the cathode 104 define a housing region 110. The material of the separator 106 is an insulating material such as polyethylene (PE), polypropylene (PP), or a composite structure (such as PE/PP/PE) formed by the above materials.

The electrolyte solution 108 is disposed in the housing region 110. The electrolyte solution 108 includes an organic solvent, a lithium salt, and an additive. The amount of the organic solvent in the electrolyte solution 108 is 55 wt % to 90 wt %, the amount of the lithium salt in the electrolyte solution 108 is 10 wt % to 35 wt %, and the amount of the additive in the electrolyte solution 108 is 0.05 wt % to 10 wt %. However, in other embodiments, the electrolyte solution 108 may also not contain an additive.

The organic solvent is, for example, γ-butyl lactone, ethylene carbonate (EC), propylene carbonate (PC), diethyl carbonate (DEC), propyl acetate (PA), dimethyl carbonate (DMC), ethylmethyl carbonate (EMC), or a combination thereof.

The lithium salt is, for example, LiPF₆, LiBF₄, LiAsF₆, LiSbF₆, LiClO₄, LiAlCl₄, LiGaCl₄, LiNO₃, LiC(SO₂CF₃)₃, LiN(SO₂CF₃)₂, LiSCN, LiO₃SCF₂CF₃, LiC₆F₅SO₃, LiO₂CCF₃, LiSO₃F, LiB(C₆H₅)₄, LiCF₃SO₃, or a combination thereof.

The additive is, for example, monomaleimide, polyrnaleimide, bismaleimide, polybismaleimide, a copolymer of bismaleimide and monomaleimide, vinylene carbonate (VC), or a mixture thereof. The monomaleimide is, for example, selected from the group consisting of N-phenylmaleimide, N-(o-methylphenyl)-maleimide, N-(m-methylphenyl)-maleimide, N-(p-methylphenyl)-maleimide, N-cyclohexylmaleimide, maleimidophenol, maleimidobenzocyclobutene, phosphorus-containing maleimide, phosphonate-containing maleimide, siloxane-containing maleimide, N-(4-tetrahydropyranyl-oxyphenyl)maleimide, and 2,6-xylylmaleimide.

The package structure 112 covers the anode 102, the cathode 104, and the electrolyte solution 108. The material of the package structure 112 is, for example, aluminum foil.

It should be mentioned that, the cathode 104 can be formed by adding the oligomer of the invention in the cathode material in a current battery manufacturing process. Therefore, the capacitance, battery efficiency, and charge and discharge cycle life of the lithium battery 100 can be effectively maintained at high temperature without modifying any battery design, electrode material, and electrolyte solution, and the lithium battery 100 can have higher safety.

In the following, the effects of the oligomer of the invention are described with experimental examples and comparative examples.

### EXPERIMENTAL EXAMPLE 1

Preparation of Anode

Metal lithium was cut into a suitable shape and inserted directly to form the anode.

Preparation of Cathode

1 part by weight (1 g) of MI was charged into a reactor loaded with 19 parts of NMP solvent, and was reacted at 80° C. for 3 hours to prepare the oligomer of MI (acting as the cathode material additive) of experimental example 1.

Next, 89 parts by weight of LiAl_{0.05}Co_{0.95}O₂, 5 parts by weight of PVDF and 5 parts by weight of acetylene black (conductive powder) were evenly mixed in the NMP solvent. Next, 1 part by weight of the oligomer of experimental example 1 was added to the mixed solution to form a cathode material. After the material was coated on an aluminum foil, the aluminum foil with the material coated thereon was dried, compressed, and then cut to form the cathode.

Preparation of Electrolyte Solution

LiPF₆ was dissolved in a mixed solution of PC, EC and DEC (volume ratio: PC:EC:DEC=2:3:5) to prepare an electrolyte solution having a concentration of 1 M, wherein the mixed solution was used as an organic solvent in the electrolyte solution and LiPF₆ was used as lithium salt in the electrolyte solution.

Manufacture of Lithium Battery

Polypropylene was used as the isolation film to isolate the anode and the cathode and define the housing region. The electrolyte solution was added into the housing region between the anode and the cathode. Lastly, the above structure was sealed with a package structure to complete the manufacture of the lithium battery of experimental example 1.

### EXPERIMENTAL EXAMPLE 2

Preparation of Anode

The anode of experimental example 2 was prepared based on the same preparation of experimental example 1.

Preparation of Cathode

1 part by weight (1 g) of BMI and TCA with a molar ratio of 3:2 were charged into a reactor loaded with 19 parts of NMP and were reacted for 8 hours at 130° C. to prepare the oligomer of TCA/BMI (acting as the cathode material additive) of experimental example 2.

Next, 89 parts by weight of LiAl_{0.05}Co_{0.95}O₂, 5 parts by weight of PVDF, and 5 parts by weight of acetylene black (conductive powder) were evenly mixed in the NMP solvent. Next, 1 part by weight of the oligomer of experimental example 2 was added to the mixed solution to form a cathode material. Then, after the material was coated on an aluminum foil, the aluminum foil with the material coated thereon was dried, compressed, and then cut to form the cathode.

Preparation of Electrolyte Solution

The procedure for preparing the electrolyte solution of experimental example 2 is the same as experimental example 1.

Manufacture of Lithium Battery

The procedure for preparing the lithium battery of experimental example 2 is the same as experimental example 1.

### EXPERIMENTAL EXAMPLE 3

Preparation of Anode

The anode of experimental example 3 was prepared based on the same preparation of experimental example 1.

Preparation of Cathode

1 part by weight (1 g) of BMI and HD with a molar ratio of 2:1 were charged into a reactor loaded with 19 parts of NMP and were reacted for 8 hours at 130° C. to prepare the oligomer of HD/BMI (acting as the cathode material additive) of experimental example 3.

Next, 89 parts by weight of LiAl_{0.05}Co_{0.95}O₂, 5 parts by weight of PVDF, and 5 parts by weight of acetylene black (conductive powder) were evenly mixed in the NMP solvent. Next, 1 part by weight of the oligomer of experimental example 3 was added to the mixed solution to form a cathode material. Then, after the material was coated on an aluminum foil, the aluminum foil with the material coated thereon was dried, compressed, and then cut to form the cathode.

Preparation of Electrolyte Solution

The procedure for preparing the electrolyte solution of experimental example 3 is the same as experimental example 1.

Manufacture of Lithium Battery

The procedure for preparing the lithium battery of experimental example 3 is the same as experimental example 1.

### EXPERIMENTAL EXAMPLE 4

Preparation of Anode

The anode of experimental example 4 was prepared based on the same preparation of experimental example 1.

Preparation of Cathode

1 part by weight (1 g) of BMA and TCA with a molar ratio of 3:2 were charged into a reactor loaded with 19 parts of NMP and were reacted for 8 hours at 130° C. to prepare the oligomer of TCA/BMA (acting as the cathode material additive) of experimental example 4.

Next, 90 parts by weight of LiAl_{0.05}Co_{0.95}O₂, 5 parts by weight of PVDF, and 5 parts by weight of acetylene black (conductive powder) were evenly mixed in the NMP solvent. Next, 1 part by weight of the oligomer of experimental example 4 was added to the mixed solution to form a cathode material. Then, after the material was coated on an aluminum foil, the aluminum foil with the material coated thereon was dried, compressed, and then cut to form the cathode.

Preparation of Electrolyte Solution

The procedure for preparing the electrolyte solution of experimental example 4 is the same as experimental example 1.

Manufacture of Lithium Battery

The procedure for preparing the lithium battery of experimental example 4 is the same as experimental example 1.

### EXPERIMENTAL EXAMPLE 5

Preparation of Anode

The anode of experimental example 5 was prepared based on the same preparation of experimental example 1.

Preparation of Cathode

1 part by weight (1 g) of BMA and HD with a molar ratio of 2:1 were charged into a reactor loaded with 19 parts of NMP and were reacted for 8 hours at 130° C. to prepare the oligomer of HD/BMA (acting as the cathode material additive) of experimental example 5.

Next, 90 parts by weight of LiAl_{0.05}Co_{0.95}O₂, 5 parts by weight of PVDF, and 5 parts by weight of acetylene black (conductive powder) were evenly mixed in the NMP solvent. Next, 1 part by weight of the oligomer of experimental example 5 was added to the mixed solution to form a cathode material. Then, after the material was coated on an aluminum foil, the aluminum foil with the material coated thereon was dried, compressed, and then cut to form the cathode.

Preparation of Electrolyte Solution

The procedure for preparing the electrolyte solution of experimental example 5 is the same as experimental example 1.

Manufacture of Lithium Battery

The procedure for preparing the lithium battery of experimental example 5 is the same as experimental example 1.

### EXPERIMENTAL EXAMPLE 6

Preparation of Anode

The anode of experimental example 6 was prepared based on the same preparation of experimental example 1.

Preparation of Cathode

1 part by weight (1 g) of BMI and THD with a molar ratio of 2:1 were charged into a reactor loaded with 19 parts of NMP and were reacted for 8 hours at 130° C. to prepare the oligomer of THD/BMI (acting as the cathode material additive) of experimental example 6.

Next, 89 parts by weight of LiAl_{0.05}Co_{0.95}O₂, 5 parts by weight of PVDF, and 5 parts by weight of acetylene black (conductive powder) were evenly mixed in the NMP solvent. Next, 1 part by weight of the oligomer of experimental example 6 was added to the mixed solution to form a cathode material. Then, after the material was coated on an aluminum foil, the aluminum foil with the material coated thereon was dried, compressed, and then cut to form the cathode.

Preparation of Electrolyte Solution

The procedure for preparing the electrolyte solution of experimental example 6 is the same as experimental example 1.

Manufacture of Lithium Battery

The procedure for preparing the lithium battery of experimental example 6 is the same as experimental example 1.

### EXPERIMENTAL EXAMPLE 7

Preparation of Anode

The anode of experimental example 7 was prepared based on the same preparation of experimental example 1.

Preparation of Cathode

1 part by weight (1 g) of BA and MI with a molar ratio of 1:1 were charged into a reactor loaded with 19 parts of NMP and were reacted for 8 hours at 100° C. to prepare the oligomer of MI/BA (acting as the cathode material additive) of experimental example 7.

Next, 89 parts by weight of LiAl_{0.05}Co_{0.95}O₂, 5 parts by weight of PVDF, and 5 parts by weight of acetylene black (conductive powder) were evenly mixed in the NMP solvent. Next, 1 part by weight of the oligomer of experimental example 7 was added to the mixed solution to form a cathode material. Then, after the material was coated on an aluminum foil, the aluminum foil with the material coated thereon was dried, compressed, and then cut to form the cathode.

Preparation of Electrolyte Solution

The procedure for preparing the electrolyte solution of experimental example 7 is the same as experimental example 1.

Manufacture of Lithium Battery

The procedure for preparing the lithium battery of experimental example 7 is the same as experimental example 1.

### COMPARATIVE EXAMPLE 1

Preparation of Anode

The anode of comparative example 1 was prepared based on the same preparation of experimental example 1.

Preparation of Cathode

1 part by weight (1 g) of BMI and barbituric acid (BTA) with a molar ratio of 2:1 were charged into a reactor loaded with 19 parts of NMP and were reacted for 8 hours at 100° C. to prepare the oligomer of comparative example 1.

Next, 89 parts by weight of LiAl_{0.05}Co_{0.95}O₂, 5 parts by weight of PVDF, and 5 parts by weight of acetylene black (conductive powder) were evenly mixed in the NMP solvent. Next, 1 part by weight of the oligomer of comparative example 1 was added to the mixed solution to form a cathode material. Then, after the material was coated on an aluminum foil, the aluminum foil with the material coated thereon was dried, compressed, and then cut to form the cathode.

Preparation of Electrolyte Solution

The procedure for preparing the electrolyte solution of comparative example 1 is the same as experimental example 1.

Manufacture of Lithium Battery

The procedure for preparing the lithium battery of comparative example 1 is the same as experimental example 1.

### COMPARATIVE EXAMPLE 2

Preparation of Anode

The anode of comparative example 2 was prepared based on the same preparation of experimental example 1.

Preparation of Cathode

The cathode of comparative example 2 was prepared according to a similar preparation process as experimental example 1, and the difference thereof is only in that: no cathode material additive was added in the cathode material of comparative example 2.

Preparation of Electrolyte Solution

The procedure for preparing the electrolyte solution of comparative example 2 is the same as experimental example 1.

Manufacture of Lithium Battery

The procedure for preparing the lithium battery of comparative example 2 is the same as experimental example 1.

Charge and Discharge Cycle Test

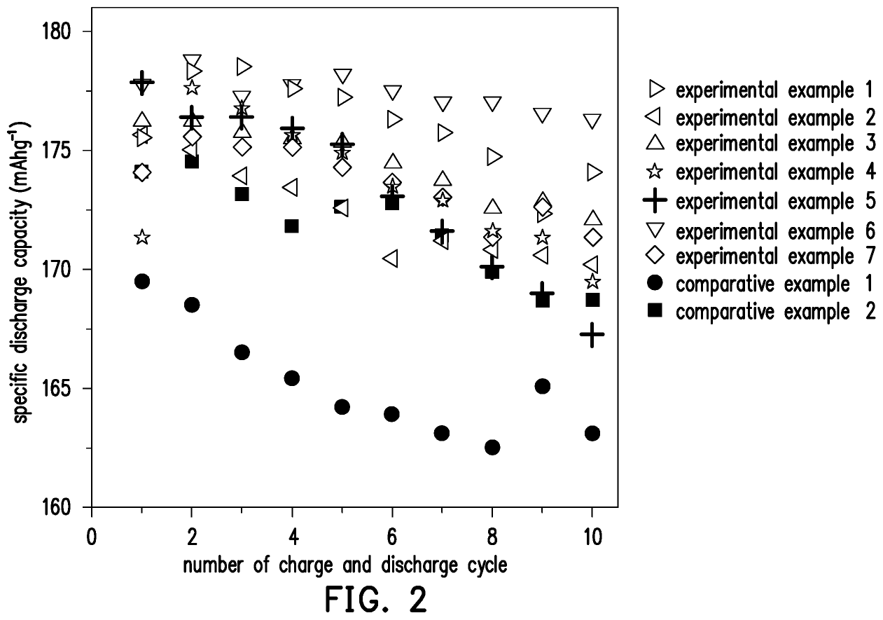

The lithium battery of each of experimental examples and comparative examples was charged and discharged at fixed current/voltage at room temperature using a potentiostat (model: VMP3), and the result is shown in FIG. 2. It can be known from FIG. 2 that the cycle life of the lithium battery of the experimental examples 1 to 7 are significantly higher than those of comparative examples 1 and 2. This indicates that the oligomer of the invention can effectively improve the battery performance. Moreover, the result also prove that the oligomer of the invention can indeed be accepted by the current lithium battery and improve the safety of the battery.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. An oligomer obtained by a polymerization reaction of a compound containing at least one ethylenically unsaturated group and a nucleophile compound, wherein the compound containing at least one ethylenically unsaturated group is selected from a group consisting of a maleimide-based compound, an acrylate ester-based compound, a methacrylate ester-based compound, an acrylamide-based compound, a vinylamide-based compound and a combination thereof, and the nucleophile compound is selected from a group consisting of monomaleimide, trithiocyanuric acid, hydantoin, hydantoin derivative, thiohydantoin, thiohydantoin derivative and a combination thereof.

2. The oligomer of claim 1, wherein a mole ratio of the compound containing at least one ethylenically unsaturated group and the nucleophile compound is between 1:5 and 5:1.

3. The oligomer of claim 1, wherein the maleimide-based compound comprises monomaleimide or bismaleimide.

4. The oligomer of claim 1, wherein the acrylate ester-based compound comprises bisphenol A diacrylate or bisphenol A ethoxylate diacrylate.

5. The oligomer of claim 1, wherein the methacrylate ester-based compound comprises bisphenol A dimethacrylate.

6. The oligomer of claim 1, wherein the acrylamide-based compound comprises bisacrylamide.

7. The oligomer of claim 1, wherein the vinylamide-based compound comprises N-vinylformamide or N-vinylacetamide.

8. The oligomer of claim 1, wherein a reaction temperature of the polymerization reaction is between 25° C. and 200° C.

9. A lithium battery, comprising:
an anode;
a cathode disposed separately from the anode, wherein the cathode comprises the oligomer of claim 1;
a separator disposed between the anode and the cathode, wherein the separator, the anode, and the cathode define a housing region;
an electrolyte solution disposed in the housing region; and
a package structure covering the anode, the cathode, and the electrolyte solution.

10. The lithium battery of claim 9, wherein the electrolyte solution comprises an organic solvent, a lithium salt, and an additive, wherein the additive comprises monomaleimide, polymaleimide, bismaleimide, polybismaleimide, a copolymer of bismaleimide and monomaleimide, vinylene carbonate, or a mixture thereof.
